(19) United States
(12) Patent Application Publication
(10) Pub. No.: US 2020/0315999 A1
(43) Pub. Date: Oct. 8, 2020

(54) TEAT DIP WITH RESIDUAL EFFICACY

(71) Applicant: DeLaval Holding AB, Tumba (SE)

(72) Inventors: Nicoleta Camelia Traistaru, Tumba (SE); Amanda Elizabeth Faltynowski, Tumba (SE); Isha Mineshkumar Patel, Tumba (SE); Carolina Mateus, Tumba (SE); Barend Jongman, Tumba (SE); Maria Buchalova, Tumba (SE); Eli Rauer, Tumba (SE); Thomas C. Hemling, Tumba (SE)

(21) Appl. No.: 16/305,017

(22) PCT Filed: May 19, 2017
(86) PCT No.: PCT/SE2017/050534

§ 371 Date: Nov. 27, 2018

(30) Foreign Application Priority Data
May 27, 2016 (SE) 1650737-8

(51) Int. Cl. A61K 31/194; A61P 31/02; A61K 33/18; A61K 9/00; A61K 47/20

(52) U.S. Cl. CPC A61K 31/194; A61P 31/02; A61K 47/20; A61K 9/0014; A61K 33/18

(57) ABSTRACT

A germicidal composition comprising at least one fast-acting germicidal agent and at least one slow-acting germicidal agent is provided. The use of a dual germicide system permits the composition to provide an immediate antimicrobial effect when applied to an animal's skin, particularly, a bovine teat, and a residual antimicrobial effect for several hours after application. A method for testing this residual antimicrobial effect is also provided.

### BACKGROUND OF THE INVENTION

### Field of the Invention

The present invention pertains to a teat dip composition comprising two or more active germicidal agents. At least one agent is specifically directed toward providing immediate disinfecting of the teat, and at least one other agent is directed toward providing ongoing germicidal activity after the teat dip is applied.

### Description of the Prior Art

Iodine is a well-known and highly efficient germicidal agent that is used for topical human an animal applications. Iodine teat dips are commonly used on farms for the prevention of mastitis. Elemental iodine is considered a broad-spectrum germicide and is capable of rapidly and completely killing bacteria on contact. Iodine solubility in water is generally low. Therefore, in order to use iodine in aqueous formulations it usually is complexed with certain polymers, glycols, or polyvinylpyrrolidone (PVP), for example. Exemplary iodine-containing formulations are disclosed in U.S. Pat. Nos. 5,618,841, 5,618,841, 5,534,266, 5,368,868, 6,852,340, RU 249008, and FR 2900795.

While iodine provides an immediate germicidal effect, it tends to lose germicidal efficacy very quickly do to its reaction with bacteria, other organic material, and its volatility. Thus, iodine does not generally provide long-lasting germicidal efficacy so that skin treated with iodine as the sole germicidal agent is subject to re-contamination shortly after treatment. Because of this, barrier teat dips have been formulated to provide for longer-lasting protection of the teat canal against environmental bacteria. These barrier teat dips generally employ film-forming polymers that create a barrier on the teat that block access to the teat canal. This barrier remains on the teat for an extended period of time, and in many cases, has to be removed before the next milking. U.S. Pat. No. 8,778,369 discloses exemplary barrier teat dip formulations.

Therefore, there is a need in the art for a germicidal teat dip formulation that provides both immediate germicidal efficacy as well as ongoing protection for several hours following application. In addition, it would be beneficial for the teat dip to not require removal prior to the next milking as is commonly required of barrier formulations.

### SUMMARY OF THE INVENTION

Embodiments of the present invention overcome one or more of the aforementioned problems with iodine-based teat dips by adding a secondary germicidal system that provides ongoing pathogen control even after the primary germicidal system has been rendered less effective or has evaporated from the animal's skin.

According to one embodiment of the present invention there is provided a germicidal composition for application to animal skin comprising at least one fast-acting germicidal agent, at least one slow-acting germicidal agent, and at least one anionic surfactant. When applied to an animal's skin contaminated with at least one pathogen, the germicidal composition causes at least a 3-log reduction in the population of the at least one pathogen within 5 minutes of application to the animal skin. The germicidal composition is also effective to prevent the re-contamination of the animal skin with the at least one pathogen for at least 4 hours following application of the germicidal composition to the animal skin.

According to another embodiment of the present invention there is provided a composition for use in methods of controlling the levels of one or more pathogens on an animal's skin comprising the germicidal compositions as described herein.

According to still another embodiment of the present invention there is provided a method of controlling the levels of one or more pathogens on an animal's skin comprising the step of applying the germicidal compositions as described herein to the animal's skin.

According to still another embodiment of the present invention there is provided a method of determining the residual germicidal efficacy of a germicidal composition including at least one fast-acting acting germicidal agent and at least one slow-acting germicidal agent. The method comprises applying the germicidal composition to a sterile test surface. The germicidal composition is then dried on the test surface, the drying step causing at least a portion of the fast-acting germicidal agent to be deactivated or removed from the germicidal composition. The test surface containing the dried germicidal composition is contacted with a microorganism-containing solution. After passage of a predetermined period of time, the test surface that has been contacted with the microorganism-containing solution is contacted with a test plate containing a microorganism growth-promoting surface. The test plate is incubated for a period of time and at a temperature sufficient for the microorganism, if present on the growth-promoting surface, to grow and be visually observable.

### BRIEF DESCRIPTION OF THE DRAWINGS

FIG. 1 is a photograph depicting growth of microorganisms on an agar test plate;

FIG. 2 is a photograph depicting moderate or some growth of microorganisms on an agar test plate; and

FIG. 3 is a photograph depicting no microorganism growth on an agar test plate.

### DETAILED DESCRIPTION OF THE PREFERRED EMBODIMENT

The present invention provides a novel approach to addressing the problem of providing longer-lasting protection of bovine teats. This approach involves employing a dual-germicide system that comprises a primary, fast-acting germicide that is capable of rapidly disinfecting the animal's teats and a secondary, slow-acting germicide that is capable of providing ongoing protection for several hours after application of the teat dip. The formulations further comprise at least one anionic surfactant.

In certain embodiments of the present invention, the germicidal composition comprises from about 0.01% to about 1.5% by weight, from about 0.1% to about 1.25% by weight, or from about 0.25% to about 1.0% by weight of the at least one fast-acting germicidal agent. As used herein, the term “fast-acting germicidal agent” generally refers to a germicidal agent that is capable of achieving at least a 5 log reduction in bacteria cell populations in 30 seconds or less. In certain embodiments, the at least one fast-acting germicidal agent is selected from the group consisting of iodine, peracetic acid, hydrogen peroxide, chlorine dioxide, sodium hypochlorite, ozonated water, sodium and potassium fluoride, hypobromous acid, chlorine, bromine, hypochlorous acid, trichloroisocyanuric acid, and combinations thereof, with iodine being a preferred fast-acting germicidal agent. The fast-acting germicidal agents, when present in the indicated amounts, are capable of effecting rapid disinfection of the animal's skin to which the composition has been applied. Iodine, in its natural form comprises long violet crystals. If the iodine crystals are heated, iodine will sublimate and transform into iodine vapors. Generally, iodine disinfectant solutions when heated, such as due to animal body temperature, will start losing iodine. Therefore, over time, the ability of the iodine to provide an adequate germicidal effect will diminish. Similar decomposition or deactivation may be observed with other species of fast-acting germicidal agents. In certain embodiments, the fast-acting germicidal agent is removed from the composition or rendered substantially inactive (i.e., incapable of achieving a 5 log reduction in bacteria cell populations in 30 seconds or less) within 2 hours after application to the animal's skin.

The slow-acting germicidal agent generally exhibits a greater stability and is, therefore, capable of providing the desired germicidal effect well after the fact-acting germicide has lost potency. As used herein, a “slow-acting germicidal agent” is generally a germicidal agent that requires at least one minute or longer to achieve a 5 log reduction in bacteria cell populations. In certain embodiments of the present invention, the at least one slow-acting germicidal agent is selected from the group consisting of C1-C12 organic acids, chlorhexidine gluconate, quaternary ammonium compounds, and combinations thereof. In particular embodiments, the at least one slow-acting is a C1-C12 organic acid selected from the group consisting of glycolic acid, lactic acid, acetic acid, propionic acid, butyric acid, heptanoic acid, octanoic acid, nonanoic acid, decanoic acid, dodecacanoic acid, oxalic acid, malic acid, malonic acid, maleic acid, fumaric acid, adipic acid, aspartic acid, glutamic acid, gluconic acid, malic acid, tartatic acid, pyruvic acid, succinic acid, benzoic acid, salicylic acid, citric acid, and combinations thereof. In preferred embodiments, the organic acid is lactic acid. Organic acids, such as the foregoing, are able to inhibit bacterial growth but generally require longer contact times to do so. Unlike the fast-acting germicides, which often can achieve a 5 log reduction from 10e7 cells/ml populations in 30 seconds or less, organic acids generally must be in contact with the microorganisms for a minute or more in order to achieve the same level of microbial control. Moreover, while the fast-acting germicidal agent used with the present invention tends not to be very stable once applied to the animal's skin, the slow-acting germicidal agent is stable. The combination of the two produces a germicidal composition having immediate and residual germicidal efficacy once applied to an animal's skin. In certain embodiments, the germicidal composition has a germicidal efficacy for at least one hour, at least 2 hours, at least 3 hours, at least 4 hours, or at least 8 hours after application to the animal's skin. In certain embodiments, the germicidal composition comprises from about 0.5% to about 10% by weight, from about 1% to about 7.5% by weight, or from about 1.5% to about 5% by weight of the at least one slow-acting germicidal agent. In certain embodiments, the weight ratio of the at least one fact-acting germicidal agent to the at least one slow-acting germicidal agent is from about 1:10 to about 10:1, or from about 1:7.5 to about 5:1, or from about 1:5 to about 2.5:1.

In certain embodiments of the present invention, both the fast-acting and slow-acting germicidal agents are broad-spectrum antimicrobials. Broad spectrum antimicrobials generally act against a wide range of microbes, particularly both Gram-positive and Gram-negative bacteria.

In order to improve the efficacy of the slow-acting germicidal agent, the germicidal compositions comprise an anionic surfactant. The anionic surfactant tends to disrupt the bacterial cell wall and cell membrane allowing for better penetration of the organic acid and easier disruption of cellular functions. In certain embodiments, the at least one anionic surfactant is selected from the group consisting of sodium lauryl sulfate, sodium octylsulfonate, sodium dioctylsulfosuccinate, sodium alpha olefin sulfonate, sodium laurylethersulfate, alkyl sulfates, alkyl aryl sulfates, alkyl aryl sulfonates, alkyl sulfonates and alcohol ethoxylates, alkyl ether sulfates, sulfated alkanol amides, disodium ricinoleate sulfate, laurylsulfoacetate and mixtures thereof. In certain embodiments, the germicidal composition comprises from about 0.05% to about 2.5% by weight, from about 0.1% to about 1.5% by weight, or from about 0.5% to about 1% by weight of the at least one anionic surfactant.

In certain embodiments of the present invention, and as illustrated by the data contained in the Examples below, the combination of fact-acting germicidal agent, slow-acting germicidal agent and anionic surfactant, in the disclosed amounts and ratios, produces a germicidal efficacy that is greater than that expected from the individual components. This synergistic germicidal effect produces rapid disinfection of the animal's skin immediately after the composition is applied as well as a residual germicidal effect for several hours after application. Surprisingly, the immediate disinfection is achieved by the fact-acting germicidal agent without a negative impact on the ability of the slow-acting germicidal agent to provide residual protection.

In certain embodiments, the germicidal composition has an acidic pH. In particular embodiments, the germicidal composition has a pH of from about 2.5 to about 5.5, or from about 3 to about 5, or from about 3.5 to about 4.5. Various pH adjusting compounds, such as sodium hydroxide, may also be added to the germicidal composition in order to achieve and maintain the desired pH. As explained below, the acidic conditions are favorable for maintaining a germicidally effective level of free iodine in the composition.

In certain embodiments, the germicidal composition has a Brookfield LV2 viscosity at 50 rpm of from about 100 to about 500 cP, or from about 150 to about 450 cP, or from about 200 to about 400 cP. Such viscosities permit the composition to be easily applied to the animal's skin, especially the animal's teats, but also to resist excessive dripping from the animal's skin before the composition can dry thereon.

The germicidal compositions may also include one or more additives such as thickeners, emollients, and iodine complexing agents. In certain embodiments, the germicidal composition further comprises from about 0.1% to about 2.5% by weight, from about 0.5% to about 2% by weight, or from about 0.75% to about 1.5% by weight of at least one thickener. Exemplary thickeners include xanthan gum, guar gum, starch and starch derivatives, for example hydroxyethyl starch or cross-linked starch, sodium alginate, carrageenan, curdlan, whey, gelatin, chitosan, chitosan derivatives, polysulfonic acids and their salts, polyacrylamide, and glycerol. Cellulosic thickeners may be used including hemicellulose, for example arabinoxylanes and glucomannanes; cellulose and derivatives thereof, for example methyl cellulose, ethyl cellulose, hydroxyethyl cellulose or carboxymethyl cellulose.

In certain embodiments, the germicidal composition further comprises from about 1% to about 20% by weight, from about 3% to about 15% by weight, or from about 5% to about 12% by weight of at least one emollient, such as glycerin, propylene glycol, sorbitol, polyethylene glycol, lanolin, ethoxylanolin, or mixtures thereof.

In certain embodiments, particularly those in which iodine is the fact-acting germicidal agent, the germicidal composition further comprises at least one iodine-complexing agent. The iodine-complexing agent increases the solubility of iodine in aqueous compositions, permitting greater levels of iodine to be introduced and remain in solution than without the use of a complexing agent. Exemplary iodine-complexing surfactants or polymers that can be used in certain embodiments of the present invention include nonylphenol ethoxylate, alcohol ethoxylates, alcohol alkoxylates, ethylene oxide-propylene oxide copolymers (such as Pluronic surfactants available from BASF, and copolymers having the general formula HO(CH₂CH₂O)ₓ(CH₂CH₂O)_{y}(CH₂CH₂O)_{z}H, wherein average x, y, and z values are 21, 67, 21, respectively), and PVP. In still other embodiments, the iodine complexing agent may comprise iodide ions. In particular embodiments, the germicidal composition comprises from about 0.5% to about 5% by weight, from about 1% to about 3.5% by weight, or from about 1.5% to about 2.5% by weight of the at least one iodine-complexing agent.

In certain embodiments, the germicidal composition may comprise iodate ion or iodide ion to provide for the chemical stability of the iodine thereby ensuring adequate levels of available iodine are maintained according to the following chemical equation:

IO₃⁻+5I⁻+6H⁺→3I₂+3H₂O.

In particular embodiments, the germicidal composition may comprise between 0 to about 1% by weight, or between about 0.001 to about 0.5% by weight, or between about 0.01 to about 0.25% by weight of iodate and/or iodide ion.

In certain embodiments of the present invention, the germicidal composition comprises less than 0.1% by weight of protein, or is substantially free of protein. Particularly, the germicidal compositions are relatively free or do not contain proteins recovered from a yeast fermentation process, such as yeast exo-proteins. Moreover, combinations of proteins and hydrogen peroxide are also avoided in this manner. In addition, certain embodiments of the present invention do not comprise chlorine release agents, namely components that when mixed together react in situ to generate a chlorine-containing antimicrobial agent such as chlorine dioxide. Accordingly, certain germicidal compositions according to the present invention comprise germicidally effective actives that are ready to use and do not require any activation or generation when added to the animal's skin.

Certain embodiments of the present invention are formulated as non-barrier teat dips. These embodiments generally do not comprise a film-forming component such as pullulan or other polysaccharide-based film forming agent such as maltodextrin or derivatives thereof. Particularly, these embodiments comprise less than 1% by weight, less than 0.5% by weight, or less than 0.1% by weight of a film-forming component. In addition, the germicidal compositions are not required to comprise a surface-functionalizing compound or component that serves as an interface between the animal's skin and the various germicidal agents, such as disclosed by US 2012/00215643, which is incorporated by reference herein in its entirety.

The germicidal agents according to the present invention provide for rapid disinfection when applied to an animal's skin, especially bovine teats, and residual efficacy for extended periods of time following application and drying of the composition on the animal's skin. In certain embodiments, when applied to an animal's skin contaminated with at least one pathogen, the germicidal composition causes at least a 3-log reduction, at least a 4-log reduction, or at least a 5-log reduction in the population of the at least one pathogen within 5 minutes, within 1 minute, or within 30 seconds of application to the animal's skin. In certain embodiments, the germicidal compositions are effective to prevent the re-contamination of the animal skin with the at least one pathogen for at least 4 hours, for at least 6 hours, or for at least 8 hours following application of the germicidal composition to the animal's skin. Exemplary pathogens capable of being controlled with the germicidal composition include E. coli, S. aureus, and S. uberis.

The germicidal compositions according to the present invention can be used in methods of controlling the levels of one or more pathogens on an animal's skin. Simply, the compositions can be applied to the animal's skin (e.g., a bovine teat) through any method known to those of skill in the art such as by dipping or spraying. Upon application, the germicidal composition, via its fast-acting germicidal component primarily, disinfects the animal's skin. After a period of time, the germicidal composition dries onto the animal's skin and the fast-acting germicidal component's efficacy in preventing re-infection of the animal's skin, such as through contact with various soils and possibly fecal matter, diminishes. However, the slow-acting germicidal component provides residual germicidal efficacy for extended periods of time, such as between washings or milking cycles.

Because the concept of residual germicidal efficacy is difficult to test under conventional test protocols, the present invention is also directed toward a microbiological test method that evaluates disinfectant efficacy of the combination of active components after a defined time post-application of the composition. This testing procedure involves the use of a Rodac (Replicate Organism Detection and Counting) plate. The agar surface of these Rodac plates form a meniscus allowing for ease of transfer of microorganisms from a contaminated surface onto the plate.

A first step in performing the test involves the preparation of the microoganisms. In certain embodiments, it is preferred that the germicidal efficacy be performed on both Staphylococcus aureus ATCC 6538 and Escherichia coli ATCC 10536. A single glycerol stock of bacteria is removed from a freezer and warmed to room temperature, or a streaked plate is obtained from a refrigerator. Plates should not be older than 4 weeks, show signs of desiccation or any other indication of loss of microbial viability. 50 ml of tryptic soy broth (TSB), or other appropriate growth media, is placed in a 125 ml flask. It is within the scope of the present invention to utilize solid growth media, such as a gel, in which to grow the bacteria and transfer it to the test surface. An inoculation loop is aseptically opened and a sample of bacteria is removed from the stock slope or a single colony is removed from the plate. The loop is used to gently submerge the bacteria into the TSB. The TSB with the bacteria is incubated at 36° C.±1° C. for at least 48 hours or until visible growth is present. This is the stock culture.

After the incubation period, a subculture of the bacteria is created by transferring 500 μl from the 48 hour culture into 50 ml of fresh TSB, or other appropriate growth media. This subculture is incubated at 36° C.±1° C. for 16-48 hours. Second and third subcultures may be prepared in the same manner, if desired. The cell suspension is then standardized to give an average of 1.0×10⁶ CFU/ml. The bacterial suspension is maintained at room temperature. If testing is to occur over a period of greater than 2 hours, the bacterial suspension is to be refrigerated in between testing samples.

The germicidal efficacy tests may be conducted at room temperature, although alternate temperatures may also be used. Discs having a diameter of approximately 22 mm are cut out of Whatman Paper Grade 4. The filter discs are sterilized using an autoclave. One or more sterile discs are placed in an empty petri dish. Germicide is added to the disc insuring that the disc is completely covered. The exact amount of germicide to be added can be varied as desired so long as the disc is completely covered. The discs are placed into an incubator at a temperature of 50° C.±1° C. over night (18-24 hr). Once dry, the discs are removed from the incubator. Using tweezers, one disc is placed into a petri dish, germicide face up. 50 μl of bacterial solution is added directly onto the disc and a timer started. Using a pipette tip, the bacteria are spread over the surface of the disc. At the designated contact time, which is typically 30 seconds (but can be adjusted as desired), the Rodac plate is placed face down onto the disc and removed immediately after contact. If the disc sticks to the Rodac plate, sterile tweezers can be used to remove it. This procedure is repeated for all samples.

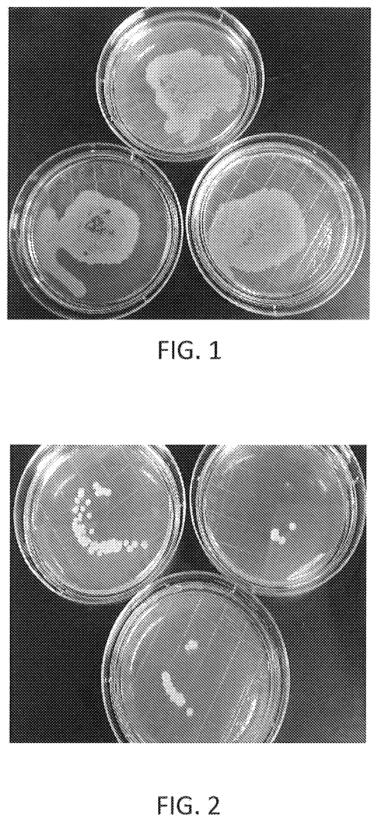

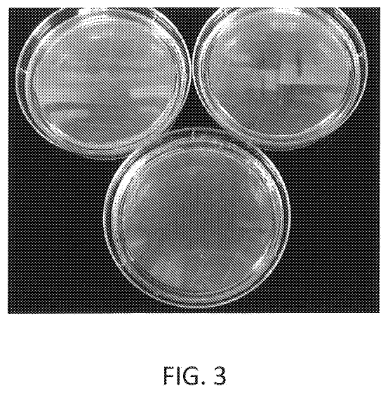

The test plates are incubated at an appropriate growth temperature for the test organism for an appropriate growth time. The level of growth is determined through visual inspection of the test plates following incubation. Exemplary test plates showing various levels of growth are provided in FIGS. 1-3. FIG. 1 depicts a plate exhibiting confluent growth of the microorganism, which indicates that the germicide did not exhibit residual efficacy. FIG. 2 depicts a test plate exhibiting some moderate level of growth, but is less than that of FIG. 1. This is indicative of some control over bacterial growth, but would be unacceptable in the field. FIG. 3 depicts a test plate having no observable bacterial growth, which indicates that the germicide tested exhibited residual efficacy. Following inspection, the plates are incubated for a second period of time to ensure that growth has not changed.

### Examples

The following examples set forth various germicidal compositions made in accordance with the present invention. It is to be understood, however, that these examples are provided by way of illustration and nothing therein should be taken as a limitation upon the overall scope of the invention.

Table 1, below, provides a number of germicidal composition formulations employing iodine as the fast-acting germicide and lactic or citric acid as the slow-acting germicide. In addition, a number of comparative formulations containing only the fast-acting or slow-acting germicides are listed.

TABLE 1

| | 1 | 2 | 3 | 4* | 5 | 6 | 7 | 8 | 9 | |
|---|---|---|---|---|---|---|---|---|---|---|
| Ingredient | % w/w | % w/w | % w/w | % w/w | % w/w | % w/w | % w/w | % w/w | % w/w | |
| Water | 82.19 | 80.53 | 77.19 | 83.77 | 82.09 | 81.99 | 81.93 | 81.83 | 81.73 | |
| Glycerin USP 99.7% | 10.03 | 10.03 | 10.03 | 10.3 | 10.03 | 10.03 | 10.03 | 10.03 | 10.03 | |
| Xanthan Gum | 0.375 | 0.375 | 0.375 | 0.375 | 0.375 | 0.375 | 0.375 | 0.375 | 0.375 | |
| polyoxyethylene, polyoxypropylene polymer | 1.71 | 1.71 | 1.71 | 0.8 | 1.81 | 1.91 | 1.71 | 1.71 | 1.71 | |
| HO(CH₂CH₂O)_{X}(CH(CH3)CH₂O)_{Y}(CH₂CH₂O)_{Z}H | | | | | | | | | | |
| in which the average value of x, y, and z are | | | | | | | | | | |
| respectively 38, 54 and 38. | | | | | | | | | | |
| polyoxyethylene, polyoxypropylene polymer | — | — | — | — | — | — | 0.26 | 0.36 | 0.46 | |
| HO(CH₂CH₂O)_{X}(CH(CH3)CH₂O)_{Y}(CH₂CH₂O)_{Z}H | | | | | | | | | | |
| average x, y, and z are 21, 67, 21 | | | | | | | | | | |
| Sodium Chloride | 0.27 | 0.27 | 0.27 | — | 0.27 | 0.27 | 0.27 | 0.27 | 0.27 | |
| Iodine | 0.59 | 0.59 | 0.59 | — | 0.59 | 0.59 | 0.59 | 0.59 | 0.59 | |
| Lactic acid 88% | 2.27 | 2.27 | 2.27 | 2.27 | 2.27 | 2.27 | 2.27 | 2.27 | 2.27 | |
| Sodium Lauryl Sulfate | 1.67 | 3.33 | 6.67 | 1.67 | 1.67 | 1.67 | 1.67 | 1.67 | 1.67 | |
| Sodium C14-C16 olefin sulfonate, 40% | — | — | — | — | — | — | — | — | — | |
| Sodium Octane Sulfonate 36% | — | — | — | — | — | — | — | — | — | |
| Alcohol Ethoxylate C9-11, 8EO | — | — | — | — | — | — | — | — | — | |
| Alcohols, C10-16, ethoxylated, sulfates, | — | — | — | — | — | — | — | — | — | |
| sodium salts 1 < EO < 2.5 25% | | | | | | | | | | |
| Sodium dioctyl sulfosuccinate, 75% | 0.05 | 0.05 | 0.05 | 0.05 | 0.05 | 0.05 | 0.05 | 0.05 | 0.05 | |
| Sodium hydroxide, 50%, FCC | 0.77 | 0.77 | 0.77 | 0.77 | 0.77 | 0.77 | 0.77 | 0.77 | 0.77 | |
| Sodium Iodide | — | — | — | — | — | — | — | — | — | |
| Sodium Iodate | 0.075 | 0.075 | 0.075 | — | 0.075 | 0.075 | 0.075 | 0.075 | 0.075 | |
| | 10 | 11 | 12 | 13 | 14 | 15 | 16 | 17 | 18 | 19 |
| Ingredient | % w/w | % w/w | % w/w | % w/w | % w/w | % w/w | % w/w | % w/w | % w/w | % w/w |
| Water | 81.99 | 81.79 | 81.69 | 81.23 | 81.73 | 80.93 | 80.43 | 80.43 | 80.43 | 82.46 |
| Glycerin USP 99.7% | 10.03 | 10.03 | 10.03 | 10.03 | 10.03 | 10.03 | 10.03 | 10.03 | 10.03 | 10.03 |
| Xanthan Gum | 0.375 | 0.375 | 0.375 | 0.375 | 0.375 | 0.375 | 0.375 | 0.375 | 0.375 | 0.300 |
| polyoxyethylene, polyoxypropylene polymer | 1.71 | 1.71 | 1.71 | 1.91 | 1.71 | 1.71 | 1.71 | 1.71 | 1.71 | 1.71 |
| HO(CH₂CH₂O)_{X}(CH(CH3)CH₂O)_{Y}(CH₂CH₂O)_{Z}H | | | | | | | | | | |
| in which the average value of x, y, and z are | | | | | | | | | | |
| respectively 38, 54 and 38. | | | | | | | | | | |
| polyoxyethylene, polyoxypropylene polymer | — | — | — | 0.26 | 0.46 | 0.46 | 0.46 | 0.46 | 0.46 | 0.46 |
| HO(CH₂CH₂O)_{X}(CH(CH3)CH₂O)_{Y}(CH₂CH₂O)_{Z}H | | | | | | | | | | |
| average x, y, and z are 21, 67, 21 | | | | | | | | | | |
| Sodium Chloride | 0.27 | 0.27 | 0.27 | 0.27 | 0.27 | 0.27 | 0.27 | 0.27 | 0.27 | 0.27 |
| Iodine | 0.59 | 0.59 | 0.59 | 0.59 | 0.59 | 0.56 | 0.56 | 0.56 | 0.56 | 0.53 |
| Lactic acid 88% | 2.27 | 2.27 | 2.27 | 2.27 | 2.27 | 2.27 | 2.27 | 2.27 | 2.27 | 1.82 |
| Sodium Lauryl Sulfate | 1.67 | 1.67 | 1.67 | 1.67 | 1.67 | — | — | — | — | 1.67 |
| Sodium C14-C16 olefin sulfonate, 40% | — | — | — | — | — | 2.50 | — | — | — | — |
| Sodium Octane Sulfonate 36% | — | — | — | — | — | — | 3.0 | — | — | — |
| Alcohol Ethoxylate C9-11, 8EO | 0.2 | 0.4 | 0.5 | 0.5 | — | — | — | — | — | — |
| Alcohols, C10-16, ethoxylated, sulfates, | — | — | — | — | — | — | — | 3.0 | — | — |
| sodium salts 1 < EO < 2.5 25% | | | | | | | | | | |
| Sodium dioctyl sulfosuccinate, 75% | 0.05 | 0.05 | 0.05 | 0.05 | 0.05 | 0.05 | 0.05 | 0.05 | 0.75 | 0.05 |
| Sodium hydroxide, 50%, FCC | 0.77 | 0.77 | 0.77 | 0.77 | 0.77 | 0.77 | 0.77 | 0.77 | 0.77 | 0.62 |
| Sodium Iodide | — | — | — | — | — | — | — | — | — | — |
| Sodium Iodate | 0.075 | 0.075 | 0.075 | 0.075 | 0.075 | 0.075 | 0.075 | 0.075 | 0.075 | 0.075 |

| | 20 | 21 | 22 | 23 | 24 | 25 | 26 | 27* | 28* |
|---|---|---|---|---|---|---|---|---|---|
| Ingredient | % w/w | % w/w | % w/w | % w/w | % w/w | % w/w | % w/w | % w/w | % w/w |
| Water | 81.26 | 82.39 | 81.78 | 81.77 | 81.86 | 81.75 | 81.72 | 83.65 | 84.74 |
| Glycerin USP 99.7% | 10.03 | 10.03 | 10.03 | 10.03 | 10.03 | 10.03 | 10.03 | 10.03 | 10.03 |
| Xanthan Gum | 0.300 | 0.300 | 0.300 | 0.300 | 0.300 | 0.300 | 0.300 | 0.300 | 0.300 |
| polyoxyethylene, polyoxypropylene polymer | 1.71 | 1.71 | 1.71 | 1.71 | 1.67 | 1.71 | 1.71 | 0.80 | 1.71 |
| HO(CH₂CH₂O)_{X}(CH(CH3)CH₂O)_{Y}(CH₂CH₂O)_{Z}H | | | | | | | | | |
| in which the average value of x, y, and z are | | | | | | | | | |
| respectively 38, 54 and 38. | | | | | | | | | |
| polyoxyethylene, polyoxypropylene polymer | 0.46 | 0.46 | 0.46 | 0.46 | 0.45 | 0.46 | 0.46 | 0.30 | 0.46 |
| HO(CH₂CH₂O)_{X}(CH(CH3)CH₂O)_{Y}(CH₂CH₂O)_{Z}H | | | | | | | | | |
| average x, y, and z are 21, 67, 21 | | | | | | | | | |
| Sodium Chloride | 0.27 | 0.27 | 0.27 | 0.27 | 0.26 | 0.27 | 0.27 | 0.00 | 0.27 |
| Iodine | 0.53 | 0..56 | 0.56 | 0.56 | 0.53 | 0.56 | 0.59 | 0.00 | 0.59 |
| Lactic acid 88% | 2.27 | 2.27 | 2.27 | 2.27 | 2.27 | 2.27 | 2.27 | 2.27 | — |
| Sodium Lauryl Sulfate | 1.67 | 1.67 | 1.67 | 1.67 | 1.67 | 1.67 | 1.67 | 1.67 | 1.67 |
| Sodium C14-C16 olefin sulfonate, 40% | — | — | — | — | — | — | — | — | — |
| Sodium Octane Sulfonate 36% | — | — | — | — | — | — | — | — | — |
| Alcohol Ethoxylate C9-11, 8EO | — | — | — | — | — | — | — | — | — |
| Alcohols, C10-16, ethoxylated, sulfates, | — | — | — | — | — | — | — | — | — |
| sodium salts 1 < EO < 2.5 25% | | | | | | | | | |
| Sodium dioctyl sulfosuccinate, 75% | 0.05 | 0.05 | 0.05 | 0.05 | 0.05 | 0.05 | 0.05 | 0.05 | 0.05 |
| Sodium hydroxide, 50%, FCC | 0.92 | 0.77 | 0.77 | 0.77 | 0.77 | 0.77 | 0.77 | 0.77 | 0.02 |
| Sodium Iodide | — | — | 0.05 | 0.05 | 0.05 | 0.05 | 0.05 | 0.05 | 0.05 |
| Sodium Iodate | 0.075 | 0.075 | 0.075 | 0.093 | 0.093 | 0.112 | 0.112 | 0.112 | 0.112 |

*Comparative formula
pH 3.6-3.9
Viscosity Lv2, 50 rpm 150-350 cP

TABLE 2a

| | | | | | | E. | S. | S. |
|---|---|---|---|---|---|---|---|---|
| Microorganism | | | | | | coli | aureus | uberis |
| For- | Formula | | | | | ATCC | ATCC | ATCC |
| mula | Concen- | Contact | | | | 10536 | 6538 | 19436 |
| # | tration | time | | Temp | Soil | LR | LR | LR |
| 1 | 80% | 30 | sec | 30° C. | Milk | 6.3 | 6.1 | 5.8 |
| 1 | 40% | 30 | sec | 30° C. | Milk | 6.3 | 6.1 | 5.8 |
| 1 | 20% | 30 | sec | 30° C. | Milk | 6.3 | 6.1 | 5.8 |
| 1 | 80% | 1 | min | 30° C. | Milk | 6.3 | 6.1 | 5.8 |
| 1 | 40% | 1 | min | 30° C. | Milk | 6.3 | 6.1 | 5.8 |
| 1 | 20% | 1 | min | 30° C. | Milk | 6.3 | 6.1 | 5.8 |
| 1 | 80% | 5 | min | 30° C. | Milk | 6.3 | 6.1 | 5.8 |
| 1 | 40% | 5 | min | 30° C. | Milk | 6.3 | 6.1 | 5.8 |
| 1 | 20% | 5 | min | 30° C. | Milk | 6.3 | 6.1 | 5.8 |

TABLE 2b

| | | | | | | E. | S. | S. |
|---|---|---|---|---|---|---|---|---|
| Microorganism | | | | | | coli | aureus | uberis |
| For- | Formula | | | | | ATCC | ATCC | ATCC |
| mula | Concen- | Contact | | | | 10536 | 6538 | 19436 |
| # | tration | time | | Temp | Soil | LR | LR | LR |
| 4 | 80% | 30 | sec | 30° C. | Milk | 1.0 | 6.1 | 2.2 |
| 4 | 40% | 30 | sec | 30° C. | Milk | 0.9 | 6.1 | 0.8 |
| 4 | 20% | 30 | sec | 30° C. | Milk | 0.1 | 2.4 | 1.2 |
| 4 | 80% | 1 | min | 30° C. | Milk | 2.9 | 6.1 | 3.7 |
| 4 | 40% | 1 | min | 30° C. | Milk | 3.0 | 6.1 | 4.4 |
| 4 | 20% | 1 | min | 30° C. | Milk | 2.3 | 5.1 | 1.8 |
| 4 | 80% | 5 | min | 30° C. | Milk | 6.3 | 6.1 | 5.8 |
| 4 | 40% | 5 | min | 30° C. | Milk | 6.3 | 6.1 | 5.8 |
| 4 | 20% | 5 | min | 30° C. | Milk | 6.3 | 6.1 | 5.8 |

Formulas 1 and 4 were selected to undergo EN1656 testing under milk soil conditions. The test data is provided in Tables 2a and 2b. The EN1656 testing occurred under conditions of contact at 30° C. for various lengths of time (ranging from 30 seconds to 5 minutes) in the presence of 1% milk solids and against the following bacteria: Escherichia coli ATCC 10536, Staphylococcus aureus ATCC 6538, and Streptococcus uberis ATCC 19463. The data shows the immediate efficacy of formula 1 due to presence of iodine. Formula 4 also shows complete kill, but the onset of efficacy is delayed to longer contact times required because of the absence of iodine.

Formulations 22, 26, and 27 were selected to undergo both EN1040 and EN1656 testing. In order to show antimicrobial efficacy under EN1040 and EN1656, a 5-log reduction in total bacteria count is required. The objective of testing under EN1040 conditions is to show general antimicrobial efficacy against the two broad bacteria classes (Gram Positive and Gram Negative). The testing under EN1040 conditions of contact at 20° C. for 30 seconds showed greater than 5 log reduction of the following two bacteria: Staphylococcus aureus ATCC 6538 and Pseudomonas aeruginosa ATCC 15442.

The EN1656 testing occurred under conditions of contact at 30° C. for 30 seconds in the presence of 1% milk solids and showed greater than 5 log reduction of the following bacteria: Escherichia coli ATCC 10536, Staphylococcus aureus ATCC 6538, and Streptococcus uberis ATCC 19463. Testing under EN1656 conditions, particularly in the presence of 1% milk solids, is intended to predict the efficacy of teat dips under field conditions, thus the elevated temperature (close to that of the udder), the presence of milk, and the short contact time.

In order to test the extended disinfectant efficacy of the germicidal compositions, the Rodac plate testing protocol, described above, was devised. The purpose of this test method is to separate the efficacy of the two different actives (fast-acting and slow-acting) in a dual germicidal product. To show this dual effect in vitro, samples of the test product were placed onto a surface and exposed to elevated temperatures for different periods of time. High temperatures increased the degradation of the fast acting germicide, iodine, while did not have an effect on the secondary germicide, lactic acid. Thus by degrading the iodine from the formula, the residual effect of lactic acid could be evaluated.

Evaluation was performed by inoculating the “aged” product with a known concentration of microorganisms. After the desired contact time, microorganisms were recovered from the surface through direct contact to an agar plate. After incubation at the appropriate temperature for 24-48 hr, microbial growth was scored as confluent (+) (see, FIG. 1), medium (±) (see, FIG. 2) or none (−) (see, FIG. 3). The advantage of this test method over traditional efficacy methods is that it closely mimics field application. The germicidal product is applied onto a surface (teat) and exposed to high microbial load (e.g. manure) for prolonged periods of time (between milkings). Traditional liquid efficacy methods, such as EN1656, expose the product to bacteria while the product is in suspension. Traditional surface efficacy methods, such as Zone of Inhibition, rely on the product migrating away from a surface, which is not the case with teat dips where the microbes land directly on the surface coated with the germicidal product and are expected to offer protection after the teat dip dries. The test method allowed for surface testing of an aged and dry product without requiring migration of the product to contact the microorganism.

Tables 3 and 4 show the efficacy of various germicidal formulations using this new test method. Each test was run in either duplicate or triplicate. The scoring (+, ±, −) is per replicate (i.e. growth on a single rodac plate). The lower the observed growth, the higher the germicidal efficacy. The tables below identify both the drying time and temperature at which the formula was dried onto the filter prior to contact with the microorganism.

Table 3 shows drying at 37° C.±1° C. for 2 to 8 hr 5 min. After the drying period the microorganisms were in contact with the active for 30 sec at 20 C±1° C. Formulas that contain the combination of lactic acid and iodine had consistently higher efficacy regardless of how long they were applied prior to contact with the organisms vs. formulas that contain only one of the actives. Efficacy was similar regardless of whether the formulations were exposed to a Gram positive organism (S. aureus) or a Gram negative organism (E. coli).

TABLE 3

| Test | | 4 | | | | | 27 | 28 |
|---|---|---|---|---|---|---|---|---|
| Con- | | (LA | | | | | (LA | (I₂ |
| ditions | 1 | only) | 14 | 21 | 25 | 26 | only) | only) |
| E. coli | −/−/− | +/+/+ | −/−/− | −/−/− | −/−/− | −/−/− | ±/±/± | −/−/− |
| 2 h 37° C. | | | | | | | | |
| S. aureus | −/−/− | ±/±/± | −/−/− | −/−/− | −/−/− | −/−/− | ±/±/± | ±/−/− |
| 2 h 37° C. | | | | | | | | |
| E. coli | −/−/− | +/+/+ | −/−/− | −/−/− | −/−/− | −/−/− | ±/±/± | −/−/− |
| 4 h 37° C. | | | | | | | | |
| S. aureus | −/−/− | +/±/± | −/−/− | −/−/− | −/−/− | −/−/− | ±/±/± | ±/−/− |
| 4 h 37° C. | | | | | | | | |
| E. coli | | | | −/−/− | −/−/− | −/−/− | ±/±/± | −/−/− |
| 6 h 37° C. | | | | | | | | |
| S. aureus | | | | −/−/− | −/−/− | −/−/− | ±/±/± | ±/±/± |
| 6 h 37° C. | | | | | | | | |
| E. coil | −/−/− | ±/+/+ | −/−/− | | | | | |
| 8 h 37° C. | | | | | | | | |
| S. aureus | −/−/− | ±/±/− | −/−/− | | | | | |
| 8 h 37° C. | | | | | | | | |

Table 4 shows drying at 50° C.±1° C. for 2 to 16 hr±5 min. After the drying period the microorganisms were in contact with the active for 30 sec at 20 C±1° C. Formulas that contain the combination of lactic acid and iodine had consistently higher efficacy regardless of how long they were applied prior to contact with the organisms vs. formulas that contain only one of the actives. Efficacy was similar regardless of whether the formulations were exposed to a Gram positive organism (S. aureus) or a Gram negative organism (E. coli).

TABLE 4

| Test | | 4 | | | | | |
|---|---|---|---|---|---|---|---|
| Conditions | 1 | (LA only) | 14 | 15 | 16 | 17 | 18 |
| E. coli | −/−/− | +/+/+ | −/−/− | | | | |
| 2 h 50° C. | | | | | | | |
| S. aureus | −/−/− | +/+/+ | −/−/− | | | | |
| 2 h 50° C. | | | | | | | |
| E. coli | −/−/− | +/+/+ | −/−/− | | | | |
| 4 h 50° C. | | | | | | | |
| S. aureus | −/−/− | −/±/± | −/−/− | | | | |
| 4 h 50° C. | | | | | | | |
| E. coli | −/−/− | +/+/+ | −/−/− | | | | |
| 8 h 50° C. | | | | | | | |
| S. aureus | −/−/− | +/+/+ | −/−/− | | | | |
| 8 h 50° C. | | | | | | | |
| E. coli | | | −/− | −/− | −/− | −/− | −/− |
| 16 h 50° C. | | | | | | | |
| S. aureus | | | −/− | −/− | −/− | −/− | −/− |
| 16 h 50° C. | | | | | | | |

1. A germicidal composition for application to animal skin comprising at least one fast-acting germicidal agent, at least one slow-acting germicidal agent, and at least one anionic surfactant, wherein, when applied to an animal's skin contaminated with at least one pathogen, the germicidal composition causing at least a 3-log reduction in the population of the at least one pathogen within 5 minutes of application to the animal skin, the germicidal composition being effective to prevent the re-contamination of the animal skin with the at least one pathogen for at least 4 hours following application of the germicidal composition to the animal skin.

2. The germicidal composition of claim 1, wherein the at least one fast-acting germicidal agent is selected from the group consisting of iodine, peracetic acid, hydrogen peroxide, chlorine dioxide, sodium hypochlorite, ozonated water, sodium and potassium fluoride, hypobromous acid, chlorine, bromine, hypochlorous acid, trichloroisocyanuric acid, and combinations thereof.

3. The germicidal composition of claim 1, wherein the germicidal composition comprises from about 0.01% to about 1.5% by weight of the at least one fast-acting germicidal agent.

4. The germicidal composition of claim 1, wherein the at least one slow-acting germicidal agent is selected from the group consisting of C1-C12 organic acids, chlorhexidine gluconate, quaternary ammonium compounds, and combinations thereof.

5. The germicidal composition of claim 4, wherein the C1-C12 organic acid is selected from the group consisting of glycolic acid, lactic acid, acetic acid, propionic acid, butyric acid, heptanoic acid, octanoic acid, nonanoic acid, decanoic acid, dodecacanoic acid, oxalic acid, maleic acid, malonic acid, maleic acid, fumaric acid, adipic acid, aspartic acid, glutamic acid, gluconic acid, malic acid, tartatic acid, pyruvic acid, succinic acid, benzoic acid, salicylic acid, citric acid and combinations thereof.

6. The germicidal composition of claim 1, wherein the germicidal composition comprises from about 0.5% to about 10% by weight of the at least one slow-acting germicidal agent.

7. The germicidal composition of claim 1, wherein the weight ratio of the at least one fact-acting germicidal agent to the at least one slow-acting germicidal agent is from about 1:10 to about 10:1.

8. The germicidal composition of claim 1, wherein the germicidal composition comprises from about 0.05% to about 2.5% by weight of the at least one anionic surfactant.

9. The germicidal composition of claim 1, wherein the at least one anionic surfactant is selected from the group consisting of sodium lauryl sulfate, sodium octylsulfonate, sodium dioctylsulfosuccinate, sodium alpha olefin sulfonate, sodium laurylethersulfate, alkyl sulfates, alkyl aryl sulfates, alkyl aryl sulfonates, alkyl sulfonates and alcohol ethoxylates, alkyl ether sulfates, sulfated alkanol amides, disodium ricinoleate sulfate, laurylsulfoacetate and mixtures thereof.

10. The germicidal composition of claim 1, wherein the germicidal composition causes at least a 5-log reduction to the at least one pathogen within 1 minute of application of the germicidal composition to the animal skin.

11. The germicidal composition of claim 1, wherein the germicidal composition has a pH of from about 2.5 to about 5.5.

12. The germicidal composition of claim 1, wherein the germicidal composition has a Brookfield viscosity using an LV2 spindle at 50 rpm of from about 100 to about 500 cP.

13. The germicidal composition of claim 1, wherein the germicidal composition further comprises from about 0.1% to about 2.5% by weight of at least one thickener.

14. The germicidal composition of claim 1, wherein the germicidal composition further comprises from about 1% to about 20% by weight of at least one emollient.

15. The germicidal composition of claim 1, wherein the germicidal composition further comprises at least one iodine-complexing agent.

16. A composition for use in methods of controlling the levels of one or more pathogens on an animal's skin comprising the germicidal composition of claim 1.

17. A method of controlling the levels of one or more pathogens on an animal's skin comprising the step of applying the germicidal composition of claim 1 to the animal's skin.

18. A method of determining the residual germicidal efficacy of a germicidal composition including at least one fast-acting acting germicidal agent and at least one slow-acting germicidal agent comprising the steps of:
applying the germicidal composition to a sterile test surface;
drying the germicidal composition on the test surface, the drying step causing at least a portion of the fast-acting germicidal agent to be deactivated or removed from the germicidal composition;
contacting the test surface containing the dried germicidal composition with a microorganism-containing solution;
after passage of a predetermined period of time, contacting the test surface that has been contacted with the microorganism-containing solution with a test plate containing a microorganism growth-promoting surface;
incubating the test plate for a period of time and at a temperature sufficient for the microorganism, if present on the growth-promoting surface, to grow and be visually observable.

19. The method of claim 18, wherein the method does not involve migration of the germicidal composition in order to contact the microorganism.
